(19) United States
(12) Patent Application Publication
(10) Pub. No.: US 2016/0081753 A1
(43) Pub. Date: Mar. 24, 2016

(54) Robot-Mounted User Interface For Interacting With Operation Room Equipment

(71) Applicant: KB Medical SA, Ecublens (CH)

(72) Inventor: Szymon Kostrzewski, Lausanne (CH)

(21) Appl. No.: 14/858,325

(22) Filed: Sep. 18, 2015

Related U.S. Application Data
(60) Provisional application No. 62/052,429, filed on Sep. 18, 2014.

(51) Int. Cl. A61B 19/00

(52) U.S. Cl. CPC A61B 19/2203; A61B 19/56; A61B 2019/2223; A61B 2019/223

(57) ABSTRACT

Described herein is robotic surgical system that includes a robot-held input device capable of communicating with operating room equipment (e.g., any equipment that a surgeon accesses during an operation). The input device is easily accessible by the surgeon as the user interface is located on the robot. The input device may be covered with a drape, such as a drape used to cover the robot or the robotic arm thereby enabling a surgeon to utilize the input device from a sterile environment.

### PRIORITY APPLICATION

This application claims priority to U.S. Provisional Patent Application No. 62/052,429, filed Sep. 18, 2014, entitled “Robot-Mounted User Interface For Interacting With Operation Room Equipment,” the content of which are hereby incorporated by reference in its entirety.

### FIELD OF THE INVENTION

Described herein are robotic surgical systems with a user interface mounted thereon for interacting with the robotic surgical system and other equipment in the operation room.

### BACKGROUND OF THE INVENTION

Robotic-assisted surgical systems have been developed to improve surgical precision and enable the implementation of new surgical procedures. For example, robotic systems have been developed to sense a surgeon's hand movements and translate them to scaled-down micro-movements and filter out unintentional tremors for precise microsurgical techniques in organ transplants, reconstructions, and minimally invasive surgeries. Other robotic systems are directed to telemanipulation of surgical tools such that the surgeon does not have to be present in the operating room, thereby facilitating remote surgery. Feedback-controlled robotic systems have also been developed to provide smoother manipulation of a surgical tool during a procedure than could be achieved by an unaided surgeon.

However, widespread acceptance of robotic systems by surgeons and hospitals is limited for a variety of reasons. Current systems are expensive to own and maintain. They often require extensive preoperative surgical planning prior to use, and they extend the required preparation time in the operating room. They are physically intrusive, possibly obscuring portions of a surgeons field of view and blocking certain areas around the operating table, such that a surgeon and/or surgical assistants are relegated to one side of the operating table. Furthermore, robotic surgical systems may interfere with a surgeon's access to other devices in the operating room. Often in the operating room there is a need to interact with various medical devices and equipment. Typically each of these systems provides its own user interface which is not user friendly. For example, the user interfaces may not be easily to sterilize or access from a sterile field due to the space occupied by a robotic surgical system. Thus, there is a need for a robotic surgical system that allows a surgeon to operate the robotic surgical system as well as other instruments in the operating room quickly and easily from a sterile field.

### SUMMARY OF THE INVENTION

Described herein is robotic surgical system that includes a robot-held input device capable of communicating with operating room equipment (e.g., any device that a surgeon accesses during an operation). The input device may be located on the robotic arm such that the input device is within working distance of a surgeon during a surgical procedure. For example, the input device may be the less than two, three, or four feet from the end effector of the robotic arm, thereby allowing a surgeon to easily utilize the robotic arm and other devices remotely controlled by the input device during a surgical procedure. Moreover, this enables a surgeon to control the robotic arm and other devices from a single input device during a surgical procedure. Additionally, the input device may be covered with a drape (e.g., a drape used to cover the robot and/or the robotic arm) such that a surgeon may utilize the input device from a sterile environment.

The disclosed technology, in certain embodiments, includes a robotic surgical system for performing surgery (e.g., spinal surgery) that includes a robotic arm with an end effector comprising a surgical instrument holder configured to securely hold a surgical instrument; an input device (e.g., a touch screen, button panel, one or more buttons, microphone, visual tracking device, gesture recognition device, motion sensor, or combination thereof) on the robotic arm for controlling the robotic surgical system and one or more operating room devices (e.g., a navigation system, medical imaging system, information system, patient data system, coagulation system, power tool, anesthesia device, and communication system); and a sterile drape configured to protect the robotic arm from contaminating a sterile field, wherein the sterile drape covers the input device thereby enabling a surgeon to utilize the input device from a sterile environment.

The robotic surgical system, in certain embodiments, includes a manipulator configured to allow robotically-assisted or unassisted positioning and/or movement of the surgical instrument by a user with at least four degrees of freedom to align an axis defined by the surgical instrument at a desired trajectory in relation to a patient situation. The manipulator may include a surgical instrument guide configured to hold and/or restrict movement of the instrument there through.

In certain embodiments, a mobile cart configured to transport the robotic surgical system. The sterile drape is configured to protect the mobile cart from contaminating a sterile field. The drape may include multiple pieces that may be connected together. For example, the sterile drape may include a first sterile drape to protect the robotic arm and a second sterile drape to protect the mobile cart.

In certain embodiments, the input device is attached to the robotic arm via a fastening apparatus, such as one or more bolts, nuts, screws, and/or electro magnets. In certain embodiments, the input device wirelessly communicates with the one or more operating room devices.

In one aspect, the disclosed technology includes a robotic surgical system for performing surgery, the system includes: a robotic arm with an end effector for securely holding and manipulating a surgical instrument; and a touch-screen input device on the robotic arm for controlling the robotic surgical system and one or more operating room devices, wherein the touch-screen input device is arranged for wired or wireless communication with a processor of the robotic surgical system and for wired or wireless communication with each of the one or more operating room devices.

In certain embodiments, the touch-screen input device is removable and operable when either attached or removed from the robotic arm.

In certain embodiments, the robotic arm comprises three joints and the touch-screen input device is located on a portion of the robotic arm between the first joint and the second joint, wherein the first joint is closer to the end effector than the second joint and the third joint.

In certain embodiments, the touch-screen input device is located on a forearm of the robotic arm.

In certain embodiments, the touch-screen input device is releasably secured to the robotic arm.

In certain embodiments, the touch-screen input device is operable when covered by a sterile drape.

In certain embodiments, the touch-screen input device is arranged for wireless communication with the processor of the robotic surgical system.

In certain embodiments, the touch-screen input device is arranged for wireless communication with each of the one or more operating room devices.

In certain embodiments, at least a portion of the touch-screen input device protrudes from the robotic arm.

In certain embodiments, the touch-screen input device is mounted flush on the robotic arm such that the touch-screen is flush with the outer surface of the robotic arm.

In certain embodiments, the system includes a sterile drape holder for securing a sterile drape over the touch-screen input device such that a surgeon can utilize the touch-screen input device from a sterile environment.

In certain embodiments, the sterile drape holder is an elastic band.

In certain embodiments, the sterile drape holder is a plastic adapter that secures a sterile drape over the touch-screen input device.

In certain embodiments, the sterile drape has a window through which a surgeon can interact with the touch-screen input device.

In certain embodiments, the system includes a sterile drape for protecting the robotic arm from contaminating a sterile field.

In certain embodiments, the sterile drape covers the input device thereby enabling a surgeon to utilize the input device from a sterile environment.

In certain embodiments, the input device is secured to the robotic arm using at least one of one or more bolts, nuts, screws, and electro magnets.

In certain embodiments, the input device is located on the robotic arm from ten inches to fifteen inches, fifteen inches to twenty inches, twenty inches to twenty five inches, or twenty five to thirty five inches away from the end effector.

In certain embodiments, the one or more operating room devices comprises at least one member selected from the group consisting of a navigation system, medical imaging system, information system, patient data system, coagulation system, power tool, anesthesia device, and communication system.

In certain embodiments, the system includes a manipulator configured to allow robotically-assisted or unassisted positioning and/or movement of the surgical instrument by a user with at least four degrees of freedom to align an axis defined by the surgical instrument at a desired trajectory in relation to a patient situation.

In certain embodiments, the manipulator comprises a surgical instrument guide configured to hold and/or restrict movement of the instrument therethrough.

In certain embodiments, the surgical instrument is a member selected from the group consisting of: a drill bit, tap, screw driver, and awl.

In certain embodiments, the robotic surgical system is for use in spinal surgery.

In certain embodiments, the system includes a mobile cart configured to transport the robotic surgical system, wherein a sterile drape is configured to protect the mobile cart from contaminating a sterile field.

In certain embodiments, the sterile drape comprises a first sterile drape to protect the robotic arm and a second sterile drape to protect the mobile cart.

In certain embodiments, the input device is attached to the robotic arm via a fastening apparatus.

In certain embodiments, the fastening apparatus is at least one member selected from the group consisting of a bolt, nut, screw, and electro magnet.

In certain embodiments, the input device wirelessly communicates with the one or more operating room devices.

In certain embodiments, the input device is located on the robotic arm with less than three feet of the end effector.

In certain embodiments, the sterile drape is transparent over at least a portion of the input device.

In certain embodiments, the system includes a force sensor located between the robotic arm and the tool holder for measuring forces and/or torques applied by a user to the first surgical tool held by the tool holder.

In certain embodiments, the system includes a handle extending from the end effector that may be grasp by a hand of a user to move and/or position the end effector.

In certain embodiments, the system includes a sensor that detects the presence of the hand of the user on the handle.

In another aspect, the disclosed technology includes a robotic surgical system, the system including: a robotic arm with an end effector comprising a surgical instrument holder configured to securely hold a surgical instrument; an input device on the robotic arm for controlling the robotic surgical system and one or more operating room devices; and a sterile drape configured to protect the robotic arm from contaminating a sterile field, wherein the sterile drape covers the input device thereby enabling a surgeon to utilize the input device from a sterile environment.

In certain embodiments, the one or more operating room devices comprises at least one member selected from the group consisting of a navigation system, medical imaging system, information system, patient data system, coagulation system, power tool, anesthesia device, and communication system.

In certain embodiments, the input device comprises at least one member selected from the group consisting of: a touch screen, button panel, one or more buttons, microphone, visual tracking device, gesture recognition device, and motion sensor.

In certain embodiments, the system includes a manipulator configured to allow robotically-assisted or unassisted positioning and/or movement of the surgical instrument by a user with at least four degrees of freedom to align an axis defined by the surgical instrument at a desired trajectory in relation to a patient situation.

In certain embodiments, the manipulator comprises a surgical instrument guide configured to hold and/or restrict movement of the instrument there through.

In certain embodiments, the surgical instrument is a member selected from the group consisting of: a drill bit, tap, screw driver, and awl.

In certain embodiments, the robotic surgical system is for use in spinal surgery.

In certain embodiments, the system includes a mobile cart configured to transport the robotic surgical system, wherein the sterile drape is configured to protect the mobile cart from contaminating a sterile field.

In certain embodiments, the sterile drape comprises a first sterile drape to protect the robotic arm and a second sterile drape to protect the mobile cart.

In certain embodiments, the input device is attached to the robotic arm via a fastening apparatus.

In certain embodiments, the fastening apparatus is at least one member selected from the group consisting of a bolt, nut, screw, and electro magnet.

In certain embodiments, the input device wirelessly communicates with the one or more operating room devices.

In certain embodiments, the input device is located on the robotic arm with less than three feet of the end effector.

In certain embodiments, the sterile drape is transparent over at least a portion of the input device.

In certain embodiments, the system includes a force sensor located between the robotic arm and the tool holder for measuring forces and/or torques applied by a user to the first surgical tool held by the tool holder.

In certain embodiments, the system includes a sensor that detects the presence of the hand of the user on the handle.

In certain embodiments, the system includes a handle extending from the end effector that may be grasp by a hand of a user to move and/or position the end effector.

In another aspect, the disclosed technology includes a method of using any of the systems described above.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1 is an illustration of an example robotic surgical system with an input device located on the robotic arm;

FIG. 2 is an illustration of an example robotic surgical system in an operating room;

FIG. 3 is an illustration of an example robotic surgical system with a sterile drape; and

FIGS. 4A and 4B are illustrations of an example system for securing a sterile drape over a user interface on a robotic arm.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DETAILED DESCRIPTION OF THE INVENTION

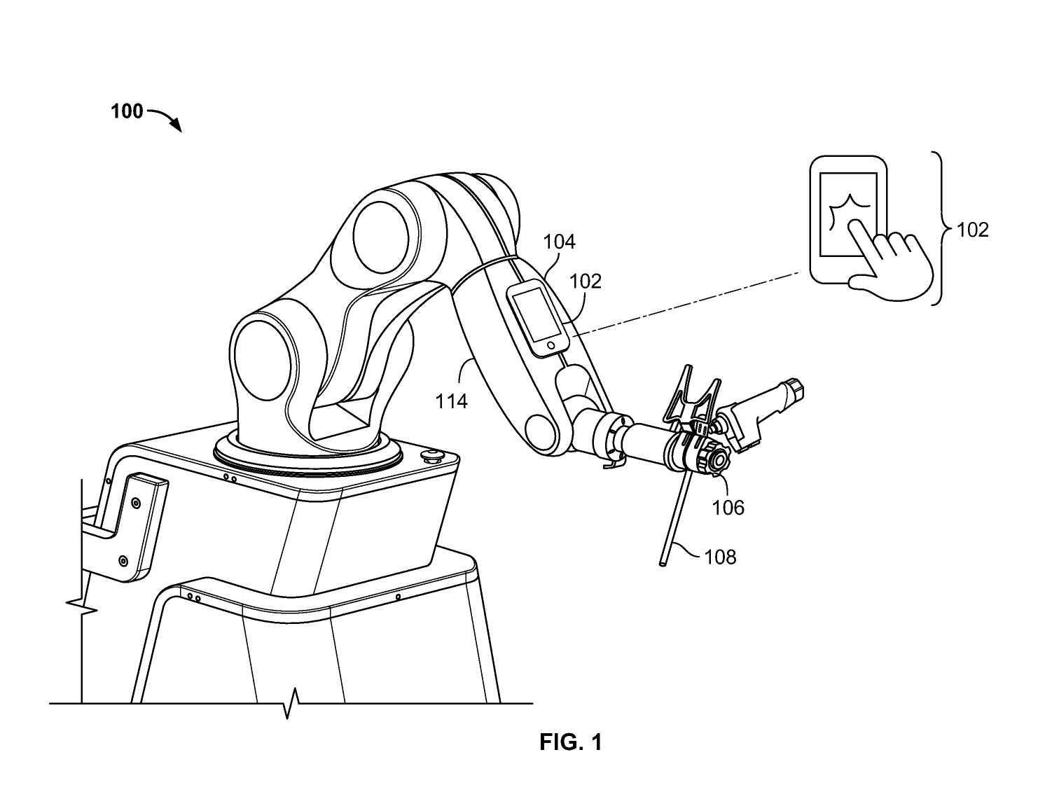

FIG. 1 is an illustration of a robotic surgical system 100 for performing surgery. During an operation, the surgeon may be guided by the robotic system to accurately execute the operation. This may be achieved by robotic guidance of the surgical tools, including ensuring the proper trajectory of the tool (e.g., drill or screw). Once the robotic arm 104 is in the desired position, the arm 104 is fixed to maintain the desired trajectory. The tool holder 106, in certain embodiments, securely holds a guide 108 through which a surgical instrument (e.g., a drill bit, tap, screw driver, awl, or other tool) may be moved through or slid at an accurate angle. Thus, the disclosed technology provides the surgeon with reliable instruments and techniques to successfully perform his/her surgery.

An input device 102 may be used to control the robotic surgical system and one or more operating room devices, such as a navigation system, medical imaging system, information system, patient data system, coagulation system, power tool, anesthesia device, and/or communication system. For example, the robotic surgical system and input device 102 may be used in spinal surgery for interacting with and/or accessing navigation system data. In another example, if the robot is guiding surgical instruments, the user interface may be used for selecting trajectories along which to guide the instruments, planning trajectories, viewing patient images, selecting surgical instrument configurations, selecting implants, making measurements, and/or controlling workflow.

In some embodiments, the input device 102 has a touch-screen. For example, the input device 102 can be a touch-screen display that acts as an extension of the computer system of the robotic surgical system. That is the input device 102 relays on computing power of the robotic surgical system (e.g., the input device 102 communicates with other systems in the operating room through the robotic surgical system).

In other examples, the input device 102 is a stand-alone touch-screen device that communications with the robotic surgical system and one or more other systems in the operating room (e.g., the input device 102 communicates directly with the other systems). In this example, the input device 102 can have a wireless communication system including an antenna (e.g., transceiver) or it can have a communication system that is hard wired to the robotic surgical system and/or one or more other systems in the operating room. In some embodiments, the input device 102 is uses wired communications with the robotic surgical system and wireless communications with one or more other surgical systems in the operating room.

In some embodiments, the input device 102 is removably fixed on the robotic arm 104 using a fastening apparatus, such as a one or more bolts, nuts, screws, and/or electro magnets. I some embodiments, the input device 102 is not removable. For example, the input device 102 can be integrated in the robotic arm 104. The input device may be located on the robotic arm such that the input device is within working distance (e.g., less than two, three, or four feet from the end effector of the robotic arm) of a surgeon during a surgical procedure. For example, the input device 102 can be located on the forearm 114 of the robotic arm 104. This enables a surgeon to control the robotic arm and other devices from a single input device during a surgical procedure. Furthermore, the input device may be covered with a drape (e.g., a drape used to cover the robot and/or the robotic arm) such that a surgeon may utilize the input device from a sterile environment. In some embodiments, the robot can optimize the configuration of the robotic arm 104 to make the input device 102 accessible (e.g., visible/reachable) for the surgeon.

In some embodiments, the input device 102 is removable and operable when either attached or removed from the robotic arm. In some embodiments, the input device 102 communicates with the operating room device(s) wirelessly. The input device 102 may be a touch screen, button panel, one or more buttons, microphone, visual tracking device, gesture recognition device, motion sensor, or combination thereof. An example robotic surgical system that may be used with the disclosed technology is described in U.S. patent Ser. No. 14/266,769, filed Apr. 30, 2014, entitled “Apparatus, Systems, and Methods for Precise Guidance of Surgical Tools,” the content of which are hereby incorporated by reference in its entirety.

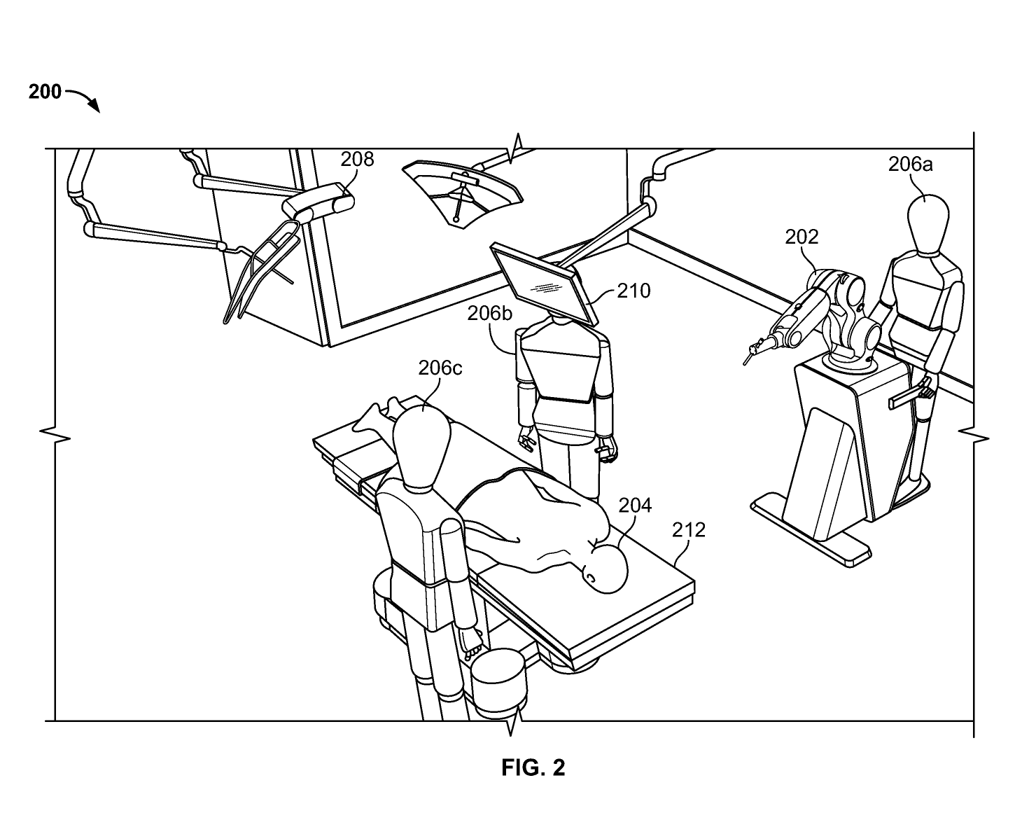

FIG. 2 illustrates an example robotic surgical system in an operating room 200. In some embodiments, one or more surgeons, surgical assistants, surgical technologists and/or other technicians, (106a-c) perform an operation on a patient 204 using a robotic-assisted surgical system. In the operating room the surgeon may be guided by the robotic system to accurately execute an operation. This may be achieved by robotic guidance of the surgical tools, including ensuring the proper trajectory of the tool (e.g., drill or screw). In some embodiments, the surgeon defines the trajectory intra-operatively with little or no pre-operative planning. The system allows a surgeon to physically manipulate the tool holder to safely achieve proper alignment of the tool for performing crucial steps of the surgical procedure. Operation of the robot arm by the surgeon (or other operator) in force control mode permits movement of the tool in a measured, even manner that disregards accidental, minor movements of the surgeon. The surgeon moves the tool holder to achieve proper trajectory of the tool (e.g., a drill or screw) prior to operation or insertion of the tool into the patient. Once the robotic arm is in the desired position, the arm is fixed to maintain the desired trajectory. The tool holder serves as a stable, secure guide through which a tool may be moved through or slid at an accurate angle. Thus, the disclosed technology provides the surgeon with reliable instruments and techniques to successfully perform his/her surgery. An example tool holder that can be used with the disclosed technology is described in U.S. patent application Ser. No. 14/695,154, filed Apr. 24, 2015, entitled “Surgical Instrument Holder for use with a Robotic Surgical System,” the content of which are hereby incorporated by reference in its entirety.

In some embodiments, the operation may be spinal surgery, such as a discectomy, a foraminotomy, a laminectomy, or a spinal fusion. In some embodiments, the surgical robotic system includes a surgical robot 202 on a mobile cart. The surgical robot 202 may be positioned in proximity to an operating table 212 without being attached to the operating table, thereby providing maximum operating area and mobility to surgeons around the operating table and reducing clutter on the operating table. In alternative embodiments, the surgical robot (or cart) is securable to the operating table. In certain embodiments, both the operating table and the cart are secured to a common base to prevent any movement of the cart or table in relation to each other, even in the event of an earth tremor.

The mobile cart may permit a user (operator) 206a, such as a technician, nurse, surgeon, or any other medical personnel in the operating room, to move the surgical robot 202 to different locations before, during, and/or after a surgical procedure. The mobile cart enables the surgical robot 202 to be easily transported into and out of the operating room 200. For example, a user 206a may move the surgical robot into the operating room from a storage location. In some embodiments, the mobile cart may include wheels, a track system, such as a continuous track propulsion system, or other similar mobility systems for translocation of the cart. The mobile cart may include an attached or embedded handle for locomotion of the mobile cart by an operator.

For safety reasons, the mobile cart may be provided with a stabilization system that may be used during a surgical procedure performed with a surgical robot. The stabilization mechanism increases the global stiffness of the mobile cart relative to the floor in order to ensure the accuracy of the surgical procedure. In some embodiments, the wheels include a locking mechanism that prevents the cart from moving. The stabilizing, braking, and/or locking mechanism may be activated when the machine is turned on. In some embodiments, the mobile cart includes multiple stabilizing, braking, and/or locking mechanisms. In some embodiments, the stabilizing mechanism is electro-mechanical with electronic activation. The stabilizing, braking, and/or locking mechanism(s) may be entirely mechanical. The stabilizing, braking, and/or locking mechanism(s) may be electronically activated and deactivated.

In some embodiments, the surgical robot 202 includes a robotic arm mounted on a mobile cart. An actuator may move the robotic arm. The robotic arm may include a force control end-effector configured to hold a surgical tool. The robot may be configured to control and/or allow positioning and/or movement of the end-effector with at least four degrees of freedom (e.g., six degrees of freedom, three translations and three rotations).

In some embodiments, the robotic arm is configured to releasably hold a surgical tool, allowing the surgical tool to be removed and replaced with a second surgical tool. The system may allow the surgical tools to be swapped without re-registration, or with automatic or semi-automatic re-registration of the position of the end-effector.

In some embodiments, the surgical system includes a surgical robot 202, a tracking detector 208 that captures the position of the patient and different components of the surgical robot 202, and a display screen 210 that displays, for example, real time patient data and/or real time surgical robot trajectories. The display screen may be located on the robotic arm. In some embodiments, the display screen 210 is a touch screen that forms the input device 102 described in relation to FIG. 1.

In some embodiments, a tracking detector 208 monitors the location of patient 204 and the surgical robot 202. The tracking detector may be a camera, a video camera, an infrared detector, field generator and sensors for electro-magnetic tracking or any other motion detecting apparatus. The tracking detector 208 may be controlled by the input device 102 as described in relation to FIG. 1. In some implementation, based on the patient and robot position, the display screen displays a projected trajectory and/or a proposed trajectory for the robotic arm of surgical robot 202 from its current location to a patient operation site. By continuously monitoring the patient and robotic arm positions, using tracking detector 208, the surgical system can calculate updated trajectories and visually display these trajectories on display screen 210 to inform and guide surgeons and/or technicians in the operating room using the surgical robot. In addition, in certain embodiments, the surgical robot 202 may also change its position and automatically position itself based on trajectories calculated from the real time patient and robotic arm positions captured using the tracking detector 208. For instance, the trajectory of the end-effector can be automatically adjusted in real time to account for movement of the vertebrae or other part of the patient during the surgical procedure.

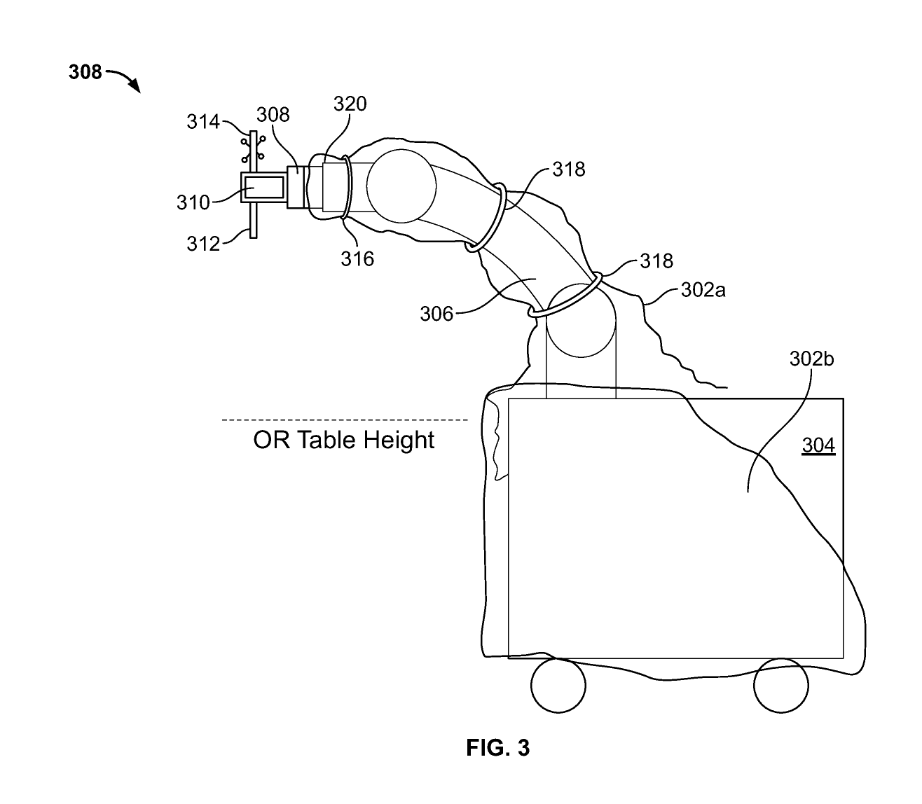

FIG. 3 illustrates an example robotic surgical system 300. In some embodiments, a sterile drape is used to protect the robotic arm from contaminating a sterile field, such as the sterile drape described in U.S. patent application Ser. No. 14/602,627, filed Jan. 22, 2015, entitled “Sterile Drape and Adapter for Covering a Robotic Surgical Arm and Preventing Contamination of a Sterile Field,” the content of which are hereby incorporated by reference in its entirety. In some embodiments, one or more surgeons, surgical assistants, surgical technologists and/or other technicians, perform an operation on a patient using a robotic-assisted surgical system. In the operating room the surgeon may be guided by the robotic system to accurately execute an operation. The robotic surgical system may be transported in and out of an operating room using a mobile cart. Accordingly, the robotic surgical system, including the mobile cart 304, must be sterilized when used in the operating room. The system may be sterilized by applying a sterile drape 302 to a portion of the system, including the robotic arm 306 and the mobile cart 304. The sterile drape may consist of a single drape or several pieces, such as sterile cover 302a for covering the robotic arm 306 and sterile drape 302b for covering the mobile cart 304. The sterile drape(s) may include printed marks configured to assist in proper draping procedure.

In some embodiments, the sterile drape 302 covers the input device on the robotic arm 306. The drape may cover the input device thereby enabling a surgeon to utilize the input device from a sterile environment. The sterile drape 302 may be transparent or a portion of the sterile drape may be transparent. For example, the sterile drape 302 may include a transparent area at least in part covering the input device.

In some embodiments, the sterile drape 302 is attached (e.g., glued or welded) to a sterile adapter 308. The sterile adapter may be attached (e.g., clipped) to an interface on the robotic arm 306. The sterile adapter ensures the drape is tightly stretched over the tool holder and robot interface to protect the robotic arm and mobile cart from contaminating the sterile field, and provides a structure that provides for repeatable and rigid positioning of the sterile drape. Tightly stretching the drape between the robot interface and tool holder and tightening the tool holder to the robotic arm using, for example, a tightening screw, reduces the likelihood of folds in the drape between the told holder and robot interface. Thus, errors between the robot model and the actual situation because of the position of the tool holder relative to the robot interface are minimized. If folds are present, the positioning of the tool holder relative to the robot interface will be different than anticipated and the robotic surgical system may have difficulty positioning tools held by the tool holder appropriately.

A tool holder 310 may be connected to the robotic arm 306 through the sterile drape. The tool holder 310 may hold sterile surgical instruments (e.g., tool guide 312) and may be coupled to a navigation marker 314. Thus, the disclosed technology enables a robotic surgical system to be used in a sterile operating room without having to sterilize each individual component of the system. Only the components outside of the sterile drape (e.g., the optical mark, surgical instruments, and tool holder must be sterilized individually.

A band 316 may be provided around the force sensor 320 mounted on the robotic arm. Band 316 may provide force sensor 320 protection. The drape may also be secured around the robotic arm using holding stripes 318. In some embodiments, the a suppression system, such as a pump, ventilator, or other suction device may be used to remove air from inside the device. In some embodiments, the holding stripes 318 are used in combination with a suppression system. In some embodiments, the sterile drape includes a drape connector configured to electrically couple, through the sterile drape, the manipulator to an electrical system of the robotic surgical system covered by the sterile drape.

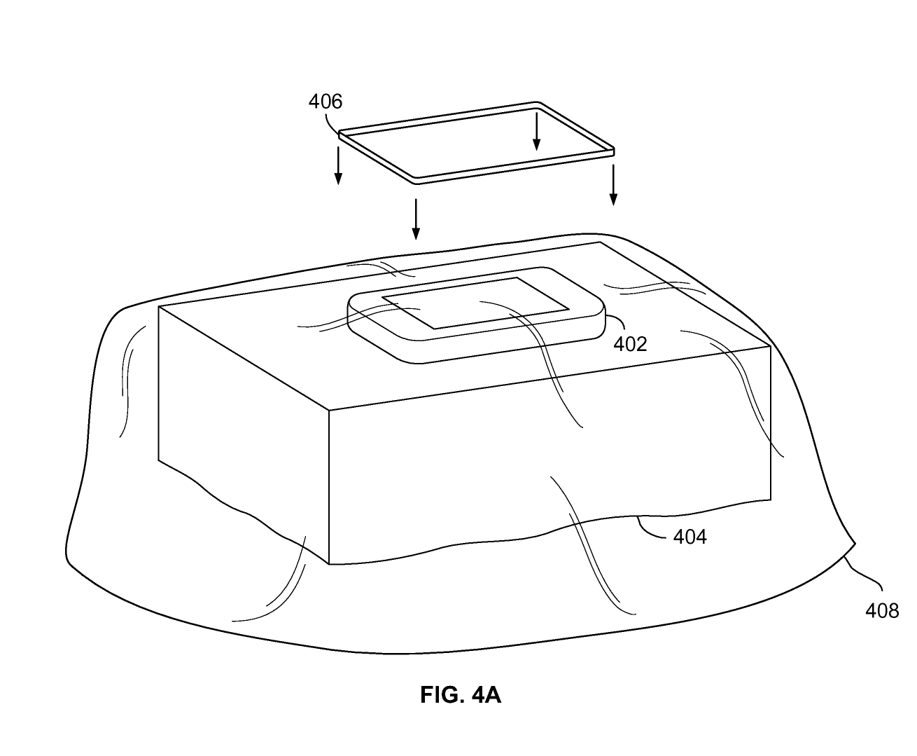

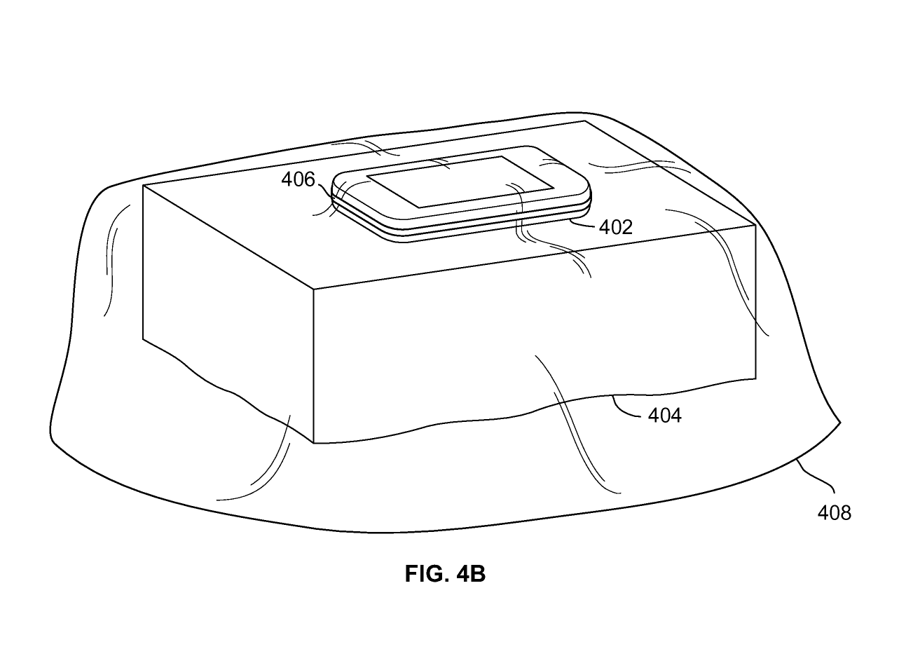

FIGS. 4A and 4B are illustrations of an example system for securing a sterile drape 408 over a input device 402 on a robotic arm 404. In certain embodiments, the input device 402 is integrated in or mounted on the robot, such as on the robotic arm 404 as illustrated in FIGS. 1, 4A, and 4B. The input device 402 can be a touch screen device. The input device 402 can be connected to the robotic surgical system with a cradle, magnet, hook-and-loop system, or other connection systems. In some embodiment, the input device 402 is releaseably connected to the robotic arm 404 (i.e., it can be removed with no tools or it can be removed with minimal tools). In other embodiments, the input device 402 is connected to the robotic arm 404 in a more permanent fashion (i.e., such that tools must be used to remove it).

In certain embodiments, the user interface 406 protrudes from the robotic arm 404. In certain embodiments, a width, a length, or both the width and length of the input device 402 increases along at least a portion of the height of the input device 402 from a base of the input device 402 that is connected to/closest to the robotic arm 404 towards the interaction surface of the input device 402 (e.g., the surface of the input device 402 a surgeon would interact with to control the robotic surgical system or other systems).

The input device 402 can be mounted on various portions of the robotic arm 404. For example, it can be mounted on the forearm of the robotic arm 404. In some embodiments, the robotic arm 404 has three joints (e.g., at least three joints) and the input device 402 is located on a portion of the robotic arm 404 between the first joint and the second joint, where the first joint is closer to the end effector than the second joint and the third joint. In certain embodiments, the input device 402 is located on the robotic arm 404 from ten inches to fifteen inches, fifteen inches to twenty inches, twenty inches to twenty five inches, or twenty five to thirty five inches away from the end effector.

While user interface 406 is described as mounted on the robotic arm 404 in this embodiment, it can be located on other parts of the robotic surgical system in other embodiments.

In certain embodiments, a sterile drape 408 is used to maintain a sterile field, such as the sterile drape described in U.S. patent application Ser. No. 14/602,627, filed Jan. 22, 2015, entitled “Sterile Drape and Adapter for Covering a Robotic Surgical Arm and Preventing Contamination of a Sterile Field,” the content of which are hereby incorporated by reference in its entirety. In certain embodiments, it is preferably to secure the sterile drape 408 over the input device 402 such that a surgeon can still interact with the input device 402 (e.g., a touch screen device) even though it is covered by the sterile drape 408. In the embodiment shown in FIGS. 4A and 4B, the sterile drape 408 is secured over the input device 402 with an elastic band 406. FIG. 4A illustrates the elastic band 406 above the input device 402 before it secures the sterile drape 408 over the input device 402. FIG. 4B illustrates the elastic band 406 after it has been installed such that it is securing the sterile drape 408 over the input device 402. The elastic band 406 allows minor adjustments to the position of the sterile drape 408 over the input device 402 to be made easily and quickly such that a tight interface is created, thereby ensuring the surgeon can interact with the input device 402 (e.g., this is particularly important in embodiments in which the input device 402 is a touch screen device). The elastic band 406 can also be sterile or sterilized easily, thereby ensuring the sterile environment is maintained.

In other embodiments, a plastic adapter is used to secure the sterile drape 408 over the input device 402. The plastic adapter, in certain embodiments, has a window that allows the surgeon to interact with the input device 402.

In certain embodiments, the sterile drape has a window sized and shaped such that the input device 402 is accessible to the surgeon. The plastic adapter can create a seal with the user interface such that the sterile field is maintained even though there is a window in the surgical drape 408. The window allows the surgeon to interact directly with the input device 402 without fear that the surgical drape 408 will interfere with the interactions.

In view of the structure, functions and apparatus of the systems and methods described here, in some embodiments, a system and method for performing surgery with a robotic surgical system are provided. Having described certain embodiments of methods and apparatus for supporting a robotic surgical system, it will now become apparent to one of skill in the art that other embodiments incorporating the concepts of the disclosure may be used. Therefore, the disclosure should not be limited to certain embodiments, but rather should be limited only by the spirit and scope of the following claims.

Throughout the description, where apparatus and systems are described as having, including, or comprising specific components, or where processes and methods are described as having, including, or comprising specific steps, it is contemplated that, additionally, there are apparatus, and systems of the disclosed technology that consist essentially of, or consist of, the recited components, and that there are processes and methods according to the disclosed technology that consist essentially of, or consist of, the recited processing steps.

It should be understood that the order of steps or order for performing certain action is immaterial so long as the disclosed technology remains operable. Moreover, two or more steps or actions may be conducted simultaneously.

1. A robotic surgical system for performing surgery, the system comprising:
a robotic arm with an end effector for securely holding and manipulating a surgical instrument; and
a touch-screen input device on the robotic arm for controlling the robotic surgical system and one or more operating room devices, wherein the touch-screen input device is arranged for wired or wireless communication with a processor of the robotic surgical system and for wired or wireless communication with each of the one or more operating room devices.

2. The robotic surgical system of claim 1, wherein the touch-screen input device is removable and operable when either attached or removed from the robotic arm.

3. The robotic surgical system of claim 1, wherein the robotic arm comprises three joints and the touch-screen input device is located on a portion of the robotic arm between the first joint and the second joint, wherein the first joint is closer to the end effector than the second joint and the third joint.

4. The robotic surgical system of claim 1, wherein the touch-screen input device is located on a forearm of the robotic arm.

5. The robotic surgical system of claim 1, wherein the touch-screen input device is releasably secured to the robotic arm.

6. The robotic surgical system of claim 1, wherein the touch-screen input device is operable when covered by a sterile drape.

7. The robotic surgical system of claim 1, wherein the touch-screen input device is arranged for wireless communication with the processor of the robotic surgical system.

8. The robotic surgical system of claim 1, wherein the touch-screen input device is arranged for wireless communication with each of the one or more operating room devices.

9. The robotic surgical system of claim 1, wherein at least a portion of the touch-screen input device protrudes from the robotic arm.

10. The robotic surgical system of claim 1, wherein the touch-screen input device is mounted flush on the robotic arm such that the touch-screen is flush with the outer surface of the robotic arm.

11. The robotic surgical system of claim 1, comprising a sterile drape holder for securing a sterile drape over the touch-screen input device such that a surgeon can utilize the touch-screen input device from a sterile environment.

12. The robotic surgical system of claim 1, wherein the sterile drape holder is an elastic band.

13. The robotic surgical system of claim 1, wherein the sterile drape holder is a plastic adapter that secures a sterile drape over the touch-screen input device.

14. The robotic surgical system of claim 1, wherein the sterile drape has a window through which a surgeon can interact with the touch-screen input device.

15-16. (canceled)

17. The robotic surgical system of claim 1, wherein the input device is secured to the robotic arm using at least one of one or more bolts, nuts, screws, and electro magnets.

18. The robotic surgical system of claim 1, wherein the input device is located on the robotic arm from ten inches to fifteen inches, fifteen inches to twenty inches, twenty inches to twenty five inches, or twenty five to thirty five inches away from the end effector.

19. The robotic surgical system of claim 1, wherein the one or more operating room devices comprises at least one member selected from the group consisting of a navigation system, medical imaging system, information system, patient data system, coagulation system, power tool, anesthesia device, and communication system.

20-25. (canceled)

26. The robotic surgical system of claim 1, wherein the input device is attached to the robotic arm via a fastening apparatus.

27. The robotic surgical system of claim 26, wherein the fastening apparatus is at least one member selected from the group consisting of a bolt, nut, screw, and electro magnet.

28-30. (canceled)

31. The robotic surgical system of claim 1, comprising a force sensor located between the robotic arm and the tool holder for measuring forces and/or torques applied by a user to the first surgical tool held by the tool holder.

32-51. (canceled)
